(19) United States
(12) Patent Application Publication
(10) Pub. No.: US 2008/0260786 A1
(43) Pub. Date: Oct. 23, 2008

(54) Process for making encapsulated particles

(76) Inventor: Daniel B. Solarek, Hillsborough, NJ (US)

Correspondence Address: NATIONAL STARCH AND CHEMICAL COMPANY, P.O. BOX 6500, BRIDGEWATER, NJ, 08807-3300, US

(21) Appl. No.: 12/112,732

(22) Filed: Apr. 30, 2008

Related U.S. Application Data
(63) Continuation-in-part of application No. 11/276,191, filed on Feb. 17, 2006.
Application No. 11/276,191 is a continuation-in-part of application No. 10/639,845, filed on Aug. 13, 2003, now Pat. No. 7,306,813.
Application No. 10/639,845 is a continuation-in-part of application No. 10/219,548, filed on Aug. 15, 2002, now Pat. No. 6,989,339.

(30) Foreign Application Priority Data
Aug. 13, 2003 (US) PCT/US03/25404

(51) Int. Cl. A61K 8/02; A61Q 5/00; C11D 17/06; A61L 9/01

(52) U.S. Cl. 424/401; 510/452; 424/76.1

(57) ABSTRACT

Method for preparing an active-containing starch matrix and the free flowing, dry powder that results from such a process. The active ingredients can be released (deposited) onto a surface by normal triggering mechanisms, which include the action of a liquid or by friction or rubbing. The fixative systems described herein provide the ability to control high load levels and protection of the active while still being able to delivery this to a substrate when triggered. Additionally, ingredients incompatible and reactive with each other can be blended together as sold/used as a one piece system and no reactions will occur until the matrix is broken or triggered.

### CROSS-REFERENCE TO RELATED APPLICATIONS

This application is a continuation-in-part of U.S. application Ser. No. 11/276 191, filed 17 Feb. 2006, which is a continuation-in-part of U.S. Pat. No. 7,306,813, filed 13 Aug. 2003, which is a continuation-in-part of U.S. Pat. No. 6,989,339, filed 15 Aug. 2002.

### BACKGROUND FIELD OF THE INVENTION

1. Technical Field

The present invention is directed towards processes for isolating dry active-containing compositions in a starch matrix. More specifically, the present invention is directed towards processes for preparing compositions having one or more active ingredients bound in a hydrocolloid matrix in dry, free flowing powder form. The present invention is also directed towards formulations containing these powdered active materials for use in personal cleansing, skin and hair care and dental products and applications.

2. Background Information

There is a need for compositions capable of containing highly mobile actives (e.g., lotions or emollients used in the treatment of skin) for ready use in consumer products. Further, there is a need for compositions and/or formulations with one or more that provide high loading of one or more actives in those consumer products.

### SUMMARY OF THE INVENTION

In order to address the above issues, the present invention provides a solution for high load of liquid or semi-solid mobile materials or actives such as fragrances, flavors, emollients, cleansing compounds such as surfactants, and skin care lotions as a dry powder form. The powder can be applied to a surface and the actives can be deposited onto a surface such as skin or a countertop by action of a trigger (e.g., water, temperature, pressure and/or friction). This is accomplished in the present invention through the use of compositions having one or more selected starches in combination and compatible with one or more actives.

In accordance with the present invention, the following definitions are used—“Active” as used herein refers to any oily mobile material (e.g., emollient, fragrance, skin care lotion or surfactant) that provides a desired benefit, such as disinfecting a surface, cleansing a surface, adding a moisturizer, biologically active, or other personal care product to skin and/or hair, etc.

“Anhydrous borax fluidity” (‘ABF’) refers to the units that the viscosity of dextrins is typically measured in. The ABF value is defined as the ratio of the amount of water to the amount of anhydrous dextrin when the latter is cooked for 5 minutes at 90° C. with 15% borax (on weight of the dextrin), so as to provide a dispersion having a viscosity of 70 mPas when cooled to 25° C. (see, e.g., U.S. Pat. No. 3,445,838).

“Emollient” as used herein refers to semi-solid or liquid material(s) used to provide a moisturizing, soothing feeling to the skin. Typical emollients suitable for this invention can be soluble or insoluble in water, and preferably are non-volatile under condition of application and use to ensure a durable effect.

“Fixed” refers to the method or process by which a mobile active such as an emollient is held in place in the dry solid matrix of starch. The loading into this matrix will depend on the nature of the active, the size of the particles and the type of starch used.

“Granular starches” refers to any starch (including chemically modified) that is in the same physical form as found in nature (e.g., not swollen or gelatinized).

“High amylose” refers to any starch or flour containing at least about 40% by weight amylose.

“Maltodextrins” refer to purified, concentrated, non-sweet nutritive mixtures of saccharide polymers obtained by partial hydrolysis of edible starch (Food Chemicals Codex, IV Edition, p. 239). Maltodextrins are generally low molecular weight versions of a base starch, whereas pyrodextrins have undergone some level of molecular rearrangement.

“Pregelatinized starches” refers to starches treated to destroy the granular structure (i.e., loss of birefringence) and swell or disperse in cold water (CWS starches).

“Pyrodextrins” refer to the hydrolysis product of starch treated at high temperature and low moisture content.

“Surfactant” refers to liquid, semi-solid or solid products used to provide compatibility between the finish and coating component in the formulation. Surfactants can also provide emulsification of the emollient and modification of the hydrophobic properties of the fibrous substrate by allowing rapid transport of aqueous liquids.

“Waxy” refers to any starch or flour containing at least about 95% by weight amylopectin.

In one embodiment the method further includes compacting the active-containing particulate to increase the particle size. In another embodiment the method includes agglomerating the active-containing particulate.

In a further embodiment the at least one fixative includes one or more starch fixatives. These one or more starch fixatives can be, for example, one or more converted starches. The one or more converted starches can be, for example, a maltodextrin and/or pyrodextrin.

In a further embodiment at least one of the one or more starch fixatives includes at least one starch modified with a reagent selected from the group consisting of organic acid anhydrides, alkylene oxides, oxidizing agents and combinations thereof In one aspect, the reagent is an organic acid anhydride. In a further aspect, the organic acid anhydride is octenyl succinic anhydride. In another aspect, the reagent is an oxidizing agent. In a further aspect, the oxidizing agent is sodium hypochloride. In even another aspect, the reagent is an alkylene oxide. In a further aspect, the alkylene oxide is propylene oxide.

In a further embodiment the at least one active containing material further includes least one surfactant. Useful surfactants include, for example, ionic, anionic, cationic, nonionic and zwitteronic surfactants. Such actives include those suitable for cleansing, disinfecting, degreasing, dispersing and so forth. The active can alternatively be, or also contain additional ingredients dissolved or suspended in the oily material (a ‘mixture of materials’), for example antioxidants, vitamins including vitamin E, medications, and the like.

### DETAILED DESCRIPTION OF THE INVENTION

In general terms, the present invention provides a formulation that enables delivery of one or more active ingredients from a free flowing, dry powdered material. Delivery to a substrate, such as hair or skin is accomplished by the action of a trigger, such as addition of water, perspiration, rubbing or temperature increase.

### Fixative—

According to the present invention, the fixative portion of the formulation includes at least one starch component. This fixative enrobes the active liquid or semi-solid and affords the free flowing powder form. In an embodiment the present invention is substantially free of fixative polymers other than the presently disclosed starch fixatives. Such other ‘fixative polymers’ include, for example, synthetic fixatives such as prepared from synthetic polymers of radical polymerization or condensation polymerization, natural and synthetic waxes, and other low molecular weight polymers.

Typical sources for starches and flours are cereals, tubers, roots, legumes and fruits. The native source or base starch can be isolated from corn, pea, potato, sweet potato, banana, barley, wheat, rice, sago, amaranth, tapioca, arrowroot, oat, canna, sorghum, cow cockle, and waxy or high amylose varieties thereof. While any starch can be useful in the practice of this invention, one embodiment of this invention includes starches isolated from corn, tapioca, sago and/or potato. In another embodiment the starches are the waxy versions of these starches.

Starches according to the present invention can be granular or pregelatinized. Also suitable are converted starches (i.e., starches wherein the molecular weight of the base starch has been reduced) derived from any of the base starch mentioned previously. These include, for example, dextrins prepared by hydrolytic action of acid and/or heat, oxidized starches prepared by treatment with oxidants such as sodium hypochlorite or hydrogen peroxide, and fluidity or thin boiling starches prepared by enzymatic conversion or mild acid hydrolysis.

The selected starch component useful in the fixative formulations of this invention can be unmodified (native) or chemically modified starches or blends of various starches. In one aspect the chemically modified starch component includes starch esters and starch ethers. Useful starch esters and/or starch ethers can contain nonionic or ionic groups such as cationic (e.g., tertiary amine and quaternary ammonium groups) or anionic groups. These starch esters and/or ether can also be crosslinked. The most suitable chemical modifications of the starch component involves treatment with organic acid anhydrides (e.g., octenyl succinic anhydride (‘OSA’)), alkylene oxides (e.g., propylene oxide (‘PO’)), and/or oxidizing reagents (e.g., sodium hypochlorite). Modified starches of these types and methods for making them are described in Starch: Chemistry and Technology, R. L. Whistler et al., Eds., Chapter X (1984).

One embodiment of these modified starches includes a starch ester prepared from an organic acid anhydride having a hydrophobic group, for example, octenyl or dodecenyl succinic anhydride. In one aspect the hydrophobic group is a hydrocarbon group such as alkyl, alkenyl, aralkyl or aralkenyl having 2 to 22 carbon atoms; in another aspect the hydrocarbon group has 5 to 18 carbon atoms; and even in another aspect the hydrocarbon group has 8 to 12 carbon atoms. Generally the starch can be treated with up to about 60% by weight of anhydride based on weight of starch in forming the starch ester. In another embodiment the starch can be treated with from about I to about 60% by weight of anhydride based on weight of starch. In even another embodiment the starch can be treated with from about 3 to about 10% by weight of anhydride based on weight of starch. A detailed description of starch ester synthesis is found in U.S. Pat. Nos. 2,661,349 and 5,672,699.

For the present invention suitable starches can be converted to water fluidity (“WF”) of at least 40. (The higher the WF the lower the molecular weight of the converted starch, and thus the lower the viscosity.) In an embodiment of this invention the starches are converted to a WF of between 40 and 70. In another embodiment the starches are either a maltodextrin or pyrodextrin.

Water fluidity measurement as described herein is made using a Thomas Rotational Shear-Type Viscometer (manufactured by Arthur H. Thomas Co., Philadelphia, Pa.) in accordance with standard procedures such as disclosed in U.S. Pat. No. 4,499,116. A further detailed description of this measurement is presented infra in the Examples section.

### Active—

Actives useful in the present invention include oily mobile materials such as emollients. Examples of commercially available classes of emollients suitable for use in the present invention include, without limitation, hydrocarbon oils and waxes, acetoglyceride esters, silicone oils, ethoxylated glycerides, triglyceride esters, alkyl and alkenyl esters, fatty acids and alcohols and their esters and ethers, lanolin and its derivatives, waxes derived from natural or synthetic sources, phospholipids and polyhydric alcohol esters. Some common examples include Aloe Vera, petrolatum, mineral oil, vitamins, antioxidants, biological actives, essential oils, hydroxy fatty acids, mono-, di- and tri-glycerides, esters and amides of fatty acids and the like. Particularly suitable emollients are mineral oil, petrolatum, vegetable oil, paraffin oil, and silicone oils. The active can also be a blend of one or more emollients and/or surfactants. The active can also contain additional ingredients dissolved or suspended in the oily material (a ‘mixture of materials’), for example medications, powders, colloidal suspensions, and the like.

In one embodiment the emollient contains a functional amount of one or more surfactants. Classes of surfactants useful for this invention are listed below. This mixture of emollient and surfactant is typically referred to as the finish. The finish can contain from 5 to 90% by weight of emollient, with the remainder being one or more surfactants.

Typically the finish is prepared by heating the solid components until all have melted, stirring until the mixture is homogenous, and then cooling with continuous stirring. The finish can be added to the fixative composition while hot or after cooling and either in undiluted form or as a dilution, usually in water.

In another embodiment, the oily mobile material of this invention is at least one or more surfactants. Useful surfactants include, for example, ionic, anionic, cationic, nonionic and zwitteronic surfactants. Non-limiting examples of surfactants suitable for use in the present invention include sulfonates of (C₁-C₂₂)alkanes and (C₂-C₂₂)alkenes; (C8-C₂₂)fatty acids of the formula R³COOH, where a mean average R³ is from about 8 to about 22 saturated or unsaturated carbon atoms and salts thereof (e.g., alkali metal, ammonium, lower alkyl amine and lower alkanol amine salts, as well as sodium, potassium, ammonium and triethanol amine); ethoxylates (2-30) of (C₈-C₂₂)fatty amines; polyoxyethylene polyols selected from sorbitol, glycerine, pentaerythritol, trimethylol ethane, trimethylol propane, and neopenyl glycol; sorbitan (C₈-C₂₂) fatty acids; ethoxylated (1-20 moles) sorbitan (C₈-C₂₂) fatty acid esters which are uncapped or capped with (C₁-C₁₀), preferably (C₁-C₄), alkoxylates; polyoxyethylene (2-100) sorbitol (C₈-C₂₂) fatty esters; ethoxylated (C₈-C₂₂) fatty alcohols having an ethylene oxide moiety corresponding to the formula —(OCH₂CH₂)ₘ, wherein m is from about 2 to about 100 moles of ethoxylation where these fatty alcohols can be straight or branched chain alcohols and can be saturated or unsaturated; phosphate and sulfonate esters of (C₈-C₂₂) fatty acids; polyalkylene oxide carboxylic acid esters having from about 8 to about 18 carbon atoms and having a polyethylene oxide moiety corresponding to the formula —(OCH₂CH₂)ₙ, where n is from about 2 to about 20, and further where mono-, di- and tri-esters are included, preferably having from about 12 to about 18 carbon atoms and where n is from about 4 to about 20; sulfonate and phosphate esters of C₁₂-C₁₈ fatty acids; sulfosuccinates; sulfosuccinamates; phenol, naphthyl, phenol (C₁-C₁₂) alkyl and naphthyl (C₁-C₁₂) alkyl sulfonates; castor oil ethoxylates (2-200 moles), and block copolymers of ethylene oxide and propylene oxide having from about 2 to about 100 moles of ethylene oxide and from about 2 to about 50 moles of propylene oxide. In another aspect, the block copolymers have from about 2 to about 50 moles of ethylene oxide. In even another aspect, the block copolymers have from about 2 to about 30 moles of propylene oxide. Examples of suitable ethoxylated fatty alcohols include oleth-, ceteth- or stearyl-2 through oleth-, ceteth- or stearyl-20, which are ethylene glycol ethers of the respective alcohols, wherein the numeric designation indicates the number of ethylene oxide moieties present and other fatty alcohols may include lauryl alcohol and isocetyl alcohol.

In one embodiment surfactants include combinations of two or more of polyoxyethylene (2-20) cetyl, stearyl or laureth alcohol, glycerol monooleate, polyoxyethylene(2-20) sorbitan (C₁₂-C₁₈) esters; and/or sorbitan (C₁₂-C₁₈)fatty acid esters. In even another embodiment the surfactants include combinations of two or more of polyoxyethylene(2) cetyl alcohol, sorbitan palmitate, polyoxyethylene(20) sorbitan monolaurate and glycerol monooleate.

### Additional Ingredients—

In addition to the fixative and the active, the formulation can optionally contain other additive ingredients normally found in such systems. Some non-limiting examples of these other ingredients include fragrances, colorants, fillers, essential oils, vitamins, disinfectants, chelating agents (e.g., EDTA, citric acid, and other organic acids), and the like.

### Preparation of Encapsulated Actives—

For the starches of this invention to be useful as encapsulants, they must be dispersed into an aqueous medium. This can be done by the traditional method of cooking the starch in a bath of boiling water, or by recent methods of steam injection or jet cooking. All of which are described in Starch: Chemistry and Technology, R. L. Whistler et al., Eds., Chapter IX (1984).

Alternately the starches of this invention can be rendered CWS (as defined above) and placed into solution by addition of the CWS powder to water with sufficient mixing. In some instances it may be advantageous to apply a small amount of heat to prepare the starch dispersion, and all effort to form a fully dispersed starch is beneficial with respect to the uniformity and loading of the final encapsulated active. The better the starch cook, the better the emulsion.

Once the starch is completely and uniformly dispersed into water, the active can be added. In the case where the active is an oil or liquid material, simple addition to the starch dispersion followed by adequate mixing will suffice. Depending on the modification of the starch, nature of the oil and if any surfactant is used will all determine the amount of shear required to form an acceptable emulsion. For purposed of this invention, the emulsion should have a mean particle size of between 1 and 100 microns. In one embodiment the emulsion will be prepared form an OSA modified starch. In another embodiment the starch is a conversion product such as pyrodextrin, maltodextrin and fluidity starch having a WF between 50 and 85.

In examples where the active is a solid or semisolid, the material must first be melted or heated to liquid form for emulsification into the starch dispersion. In an embodiment of these type of active materials are chosen from the group consisting of waxes and petrolatum. In some examples it may even be necessary to warm the starch dispersion to keep the solid or semi-solid active from crystallizing during processing.

The emulsion of the active in the starch dispersion should generally be done at the highest solids possible to maximize through-put in the drier and reduce costs. The use of converted starches and low molecular weight materials such as maltodextrin allow for easy handling at these higher solids. In one embodiment of this invention the emulsion is prepared at total solids of between 10 and 70 percent. In another embodiment, total solids is between 30 and 60 percent.

Loading of active (the amount contained within the starch robe) should also be as high as possible. However, too high a loading can provide a thin weak starch robe and the potential for leakage of active, while too low a loading can be ineffective at delivering active to the surface of the application considered. In one embodiment of this invention active loading in the starch matrix is between 10 and 70 percent. In another embodiment active is present in the starch matrix at between 30 and 60 percent.

Wall thickness of the starch robe can act as a variable release mechanism. Thin robes give fast release, while thicker robes provide slower, longer lasting release.

Once the emulsion has been formed with the aqueous starch dispersion, the next step is to dry the product. This can be accomplished by methods typically know in the art, such as spray drying, drum drying and freeze drying. In one embodiment of this invention the dry free flowing product containing the active ingredient is prepared by spray drying the emulsion. In an embodiment of this invention the moisture content of the free flowing powder is between 3 and 15% moisture.

After being subjected to spray-drying or drum-drying, the free flowing powder product may optionally be agglomerated. Agglomeration may be achieved by methods known in the art, including, for instance, via batch or continuous processing. A particularly useful method of agglomeration involves spraying the material recovered from the spray tower with water until the individual particles adhere to one another. The particles are then dried with heated air to final moisture content of from about 3% to about 12%.

The dried powders can be compacted using any means known in the art. A particularly useful method of compacting is by feeding the powder through a roller compactor, such as a chilsonator. After the initial spray-drying or drum-drying wherein the active is encapsulated, the dried powder is subjected to compact granulation or chilsonation in order to build (increase) particle size. No additional moisture is required for this process. Useful particle ranges of interest include conditions wherein approximately 70% of the particles are within the range of about 700 to 800 microns, with the remaining 30% divided above and below this range. In another embodiment, approximately 70% of the particles are within the range of about 1200 microns, with the remaining 30% divided above and below this range. In another embodiment, approximately 70% of the particles are within the range of about 2000 microns, with the remaining 30% divided above and below this range. Compaction as well as other methods for changing the particle size distribution can and often will change the rate and time of release of the material held within the starch matrix.

Another useful method of compacting is by extrusion. When extrusion is used, starch can become pregelatinized and compacted during the same process. The particle size of compacted CWS starch powders can be reduced by methods known in the art such as milling. The particle size distribution of the powders can also be optionally narrowed using methods known in the art such as sieving. The roller compaction process can also be combined with milling and sieving processes to again obtain a precise particle size distribution.

Use of these powders to deliver active to many different surfaces are incorporated in the invention. Some non-limiting examples of uses for the dry free flowing powder is as body powder, hard surface cleaner, hair or skin treatment, deodorizers for personal care, room freshening deodorizers, permanent press applications, deposition of actives to oral and dental surfaces, deposition of hair colorants, fabric care such as stain prevention, first aid and medical treatment and many other personal care and household applications.

### EXAMPLES

The following examples are presented to further illustrate and explain the present invention and should not be taken as limiting in any regard. Unless stated otherwise, all percents are in a weight/weight basis.

### Water Fluidity Measurement

Starch water fluidity (‘WF’) is measured using a Thomas Rotational Shear-Type Viscometer (manufactured by Arthur H. Thomas Co., Philadelphia, Pa. 19106), standardized at 30° C. with a standard oil having a viscosity of 24.73 mPas, requiring 23.12+/−0.05 seconds for 100 revolutions. Accurate and reproducible measurements of WF are obtained by determining the time which elapses for 100 revolutions at different solids levels depending on the starch's degree of conversion (as the degree of conversion increases, WE increases and viscosity decreases). The procedure used involves slurrying the required amount of starch (e.g., 6.16 g, dry basis) in 100 ml of distilled water in a covered copper cup and heating the slurry in a boiling water bath for 30 minutes with occasional stirring. The starch dispersion is then brought to the final weight (e.g., 107 g) with distilled water. The time required for 100 revolutions of the resultant dispersion at 81-83° C. is recorded and converted to a water fluidity number using a conversion table.

TABLE 1
WF Measurements
Time required for 100 Revolutions (seconds)
Amount of Starch Used (anhydrous, g):

| | | | | Water |
|---|---|---|---|---|
| 6.16^{a} | 8.80^{b} | 11.44^{c} | 13.20^{d} | Fluidity |
| 60.0 | | | | 5 |
| 39.6 | | | | 10 |
| 29.3 | | | | 15 |
| 22.6 | | | | 20 |
| 20.2 | | | | 25 |
| | 33.4 | | | 30 |
| | 27.4 | | | 35 |
| | 22.5 | | | 40 |
| | 32.5 | | 45 | |
| | 26.8 | | 50 | |
| | 22.0 | | 55 | |
| | | 24.2 | 60 | |
| | | 19.2 | 65 | |
| | | 15.9 | 70 | |
| | | 13.5 | 75 | |
| | | 11.5 | 80 | |
| | | 10.0 | 85 | |
| | | 9.0 | 90 | |

For a, b, c and d, final weight of each starch solutions is 107, 110, 113 and 115 g, respectively.

### Example 1

### Dextrin Fixative for Water Insoluble Emollient and Fragrance

This illustrates the production of an emollient emulsion, the spray application of that emulsion to form a dry fee flowing powder.

A pyrodextrin produced from tapioca starch with an ABF of about 4 and that had been treated with about 3% octenyl succinic anhydride was slurried in water and cooked by direct steam injection in a model C-1 jetcooker (National Starch and Chemical Company, Bridgewater, N.J.) to produce a dextrin dispersion at about 45 percent anhydrous solids. About 300 ml of this dispersion at 49° C. (120° F.) was placed into a one liter 316 stainless steel beaker and mixed with a Silverson model L4RT laboratory emulsifier (Silverson Machines, Inc., East Longmeadow, Mass.) fitted with a 31.75 mm (1.25 inch) diameter fine screen emulsifying head. The mixer speed was set at 10,000 rpm. Sufficient Dow Coming 245 silicone oil (Dow Coming, Midland, Mich.) was slowly added over a five minute period to give an anhydrous ratio of 45 parts dextrin and 25 parts Dow Corning oil. Aloe Vera extract (Verogel 1:1, Dr. Madis Laboratories, South Hackensack, N.J.) was added to the dispersion with mixing in an amount sufficient to give an anhydrous ratio of 45 parts dextrin, 25 parts silicone oil and 25 parts aloe extract. Peppermint oil (redistilled peppermint oil FFC obtained from Ungerer Co., Lincoln Park, N.J.) was added to the dispersion with mixing in an amount sufficient to give an anhydrous ratio of 45 parts dextrin, 25 parts silicone oil, 25 parts aloe extract and 5 parts peppermint oil. This was diluted with warm water to about 20% solids and then spray-dried to a free flowing powder.

### Example 2

### Screening Evaluation for Various Starch-Based Fixatives

Dextrin dispersions were prepared as described in Example 1 and diluted to 20%. The cooked starches were then blended with Atphos® MBA 1310 and polyoxyethylene Lial 125 (C₁₂-15) alcohol at the specified anhydrous ratio and were blended with a Silverson model L4RT laboratory emulsifier (Silverson Machines, East Longmeadow, Mass.) fitted with a 31.75 mm (1.25 inch) diameter fine screen emulsifying head for about 5 minutes. These mixtures were drawn on a glass plate as a 0.254 mm (0.01 inch) wet film and dried at room temperature for 24 hours.

TABLE 2
screening of various starches for use as emollient fixatives

| | | Starch/ | | |
|---|---|---|---|---|
| | Formulation | Emollient | | |
| Starch | Solids | Ratio | Appearance | Evaluation |
| Waxy maize, | 10% solids | 1:1 | Acceptable | Separate |
| WF = 40, 3% OSA | | | Viscosity | oily film |
| Waxy maize, | 20% | 1:1 | Acceptable | No |
| WF = 70, 3% OSA | | | Viscosity | separation |
| Waxy maize, | 20% | 1:1 | Acceptable | No |
| WF = 85, 3% OSA | | | Viscosity | separation |
| Waxy maize, | 10% solids | 2:3 | Acceptable | Separate |
| WF = 40, 3% OSA | | | Viscosity | oily film |
| Waxy maize, | 20% | 2:3 | Acceptable | Separate |
| WF = 70, 3% OSA | | | Viscosity | oily film |
| Waxy maize, | 20% | 2:3 | Acceptable | Slight |
| WF = 85, 3% OSA | | | Viscosity | Separation |
| Waxy maize, | 10% | 1:1 | Too thick | No |
| cross-linked | | | | separation |
| Potato | 10% | 1:1 | Too thick | Sep |
| | | | | oily film |
| Canary corn dextrin, | 20% | 1:1 | Acceptable | No |
| ABF = 2 | | | Viscosity | separation |
| Canary corn dextrin, | 20% | 2:3 | Acceptable | Separate |
| ABF = 2 | | | Viscosity | oily film |

“Appearance” shows observations of the wet mixture at specified solids; too thick could not be readily sprayed or roll coated (typically greater than 1000 mPas). This demonstrates that a certain minimum level of conversion (hydrolysis of the base starch or reduction of the molecular weight) is desirable for most applications.

“Evaluation” shows observations of the dry films and predicts the ability of these mixtures to fix the emollient in a dry particle. Oil separation shows that the starch and surfactant are not compatible and will not be able to hold (fix) the emollient onto the surface of the web.

This example also shows that certain starches, while being useful as a matrix for the emollient, may be too viscous to make application practical. Likewise, low viscosity starches may be suitable for application but may not function acceptably in forming the active matrix, especially at higher loadings.

### Example 3

### Oily Mobile Formulation

This example illustrates production of a surfactant-containing composition and creation of a dry free flowing powder containing the oily mobile phase within the starch matrix.

Acid-hydrolyzed amylopectin chemically modified with 1-octenyl butane dioate was added to 0.833 kg of water to form a 2.083 kg 30% solids starch solution. 1.250 kg of a 50% active quaternary ammonium cationic surfactant (commercially available as Barquat® 4250-Z, a quaternary ammonium disinfectant with alkyl dimethyl benzyl ammonium chloride and alkyl dimethyl ethylbenzyl ammonium chloride as the active ingredients, from Lonza Ltd., Basel, Switzerland) was added to the solution. The solution was mixed and heated to 40-45° C. and spray-dried at an inlet nozzle temperature of about 143-166° C. (290-330° F.) and outlet nozzle temperature of about 74-82° C. (165-180° F.). An off-white, yellowish free-flowing powder of 50 parts dextrin to 50 parts surfactant was produced.

### Example 4

### Oily Mobile Formulation

This example illustrates production of a surfactant-containing composition useful for applying to non-woven webs and its application thereto.

3.90 kg of a 100% active nonionic surfactant (commercially available as Triton® DF-12, a low foam nonionic surfactant, from The Dow Chemical Company, used in household cleansing as, for example, a defoamer or degreaser) was added with mixing to 5.60 kg of water. The solution was then added to a 10.0 kg 39% solids starch solution (amylopectin chemically modified with 1-octenyl butane dioate). The solution (40% solids) was mixed and spray-dried at an inlet nozzle temperature of about 191-196° C. (376-385° F.) and outlet nozzle temperature of about 93-99° C. (200-210° F.). An off-white powder of 50 parts modified starch to 50 parts surfactant was produced.

### Example 5

### Oily Mobile Formulation

This example illustrates production of a surfactant-containing composition useful for applying to non-woven webs and its application thereto.

3.50 kg of a 100% active nonionic surfactant (commercially available as Triton® DF-12, a low foam nonionic surfactant, from The Dow Chemical Company) was added with mixing to 12.70 kg of water. The solution was then added to a 10.0 kg 35% solids starch solution (acid-hydrolyzed amylopectin chemically modified with 1-octenyl butane dioate). The solution (27% solids) was mixed and spray-dried at an inlet nozzle temperature of about 191-196° C. (376-385° F.) and outlet nozzle temperature of about 93-99° C. (200-210° F.). An off-white powder of 50 parts modified starch and 560 parts surfactant was produced.

### Example 6

### Oily Mobile Formulation

This example illustrates production of a surfactant-containing composition useful for applying to non-woven webs and its application thereto.

Acid-hydrolyzed amylopectin chemically modified with 1-octenyl butane dioate was added to water to form a starch solution. A 50% active alkyl polyglycoside nonionic surfactant (commercially available as APG®, a wetting and dispersing surfactant consisting of a hydrophilic saccharide moiety and a hydrophobic fatty alkyl chain, from Cognis, Düsseldorf, Germany) was added to the solution with mixing to form a 21-27% solids solution. The solution was spray-dried at an inlet nozzle temperature of about 177° C. (350° F.) and outlet nozzle temperature of about 88° C. (190° F.). An off-white, yellowish free-flowing powder of 50 parts dextrin to 50 parts surfactant was produced.

The resultant powder was further chilsonated. Three cuts of the powder centering around 800, 1200 and 2000 microns were taken.

### Example 7

### Oily Mobile Formulation

This example illustrates production of an enzyme-containing composition useful for applying to non-woven webs and its application thereto.

Acid-hydrolyzed amylopectin chemically modified with 1-octenyl butane dioate was added to water to form a 1.035 kg starch solution of 29% solids. 0.653 kg ethylene diamine tetra acetic acid (‘EDTA’) (commercially available as Sequestrene K4 EDTA bulk, from CIBA-Geigy, Basel, Switzerland) was added to the solution with mixing to form a 35.2% solids solution. The solution was spray-dried at an inlet nozzle temperature of about 200° C. (392° F.) and outlet nozzle temperature of about 120° C. (248° F.). A yellowish free-flowing powder of 50 parts dextrin to 50 parts EDTA was produced.

The resultant powder was further chilsonated. Three cuts of the powder centering around 800, 1200 and 2000 microns were taken.

### Example 8

### Oily Mobile Formulation

This example illustrates production of a surfactant-containing composition useful for applying to non-woven webs and its application thereto.

Amylopectin chemically modified with 1-octenyl butane dioate was added to water to form a starch solution. An alkyl diphenyloxide disulfonate anionic surfactant (commercially available as Dowfax 2A1®from The Dow Chemical Company, Midland, Mich.), useful in cleaning, was added to the solution with mixing to form a solution. The solution was spray-dried at an inlet nozzle temperature of about 191-196° C. (376-385° F.) and outlet nozzle temperature of about 93-99° C. (200-210° F.). An off-white powder of 50 parts dextrin to 50 parts surfactant was produced.

The resultant powder was further chilsonated. Three cuts of the powder centering around 800, 1200 and 2000 microns were taken.

### Example 9

### Oily Mobile Formulation

This example illustrates production of a surfactant-containing composition useful for applying to non-woven webs and its application thereto.

Acid-hydrolyzed amylopectin chemically modified with 1-octenyl butane dioate was added to water to form a starch solution. A 50% active alkyl dimethyl benzyl ammonium chloride nonionic surfactant (commercially available as Barquat MB-50®, a quaternary ammonium compound, from Lonza Ltd., Basel, Switzerland), useful as a sanitizer or disinfectant, was added to the solution with mixing. The solution was spray-dried at an inlet nozzle temperature of about 143-166° C. (290-330° F.) and outlet nozzle temperature of about 74-82° C. (165-180-F). Off-white, yellowish free-flowing powder of 50 parts dextrin to 50 parts surfactant was produced.

The resultant powder was further chilsonated. Three cuts of the powder centering around 800, 1200 and 2000 microns were taken.

Although the present invention has been described and illustrated in detail, it is to be understood that the same is by way of illustration and example only, and is not to be taken as a limitation. The spirit and scope of the present invention are to be limited only by the terms of any claims presented hereafter.

What is claimed is:

1. A method of preparing a dry free flowing powder, the method comprising:
preparing an emulsion of at least one active-containing material and one or more starch materials; and
spray-drying the emulsion, thereby forming an active-containing particulate in the form of a dry, free flowing powder

2. Method according to claim 1 further comprising the step of adding an emulsifying agent to the emulsion.

3. Method according to claim 1 further comprising the step of compacting the active-containing particulate.

4. Method according to claim 1 further comprising the step of agglomerating the active-containing particulate.

5. Method according to claim 1 wherein at least one of the one or more starch materials is one or more converted starches.

6. Method according to claim 4 wherein at least of the one or more starch materials is modified with a reagent that renders the starch material cationic.

7. Method according to claim 4 wherein the one or more starch materials is chosen from pyrodextrins, maltodextrins and fluidity starches of WF between 50 and 85.

8. Method according to claim 4 wherein at least one of the one or more starch materials is a starch material modified with a reagent chosen from organic acid anhydrides, alkylene oxides, oxidizing agents and combinations thereof.

9. Method according to claim 6 wherein the reagent is an organic acid anhydride.

10. Method according to claim 6 wherein the organic acid anhydride is octenyl succinic anhydride.

11. Method according to claim 6 wherein the organic acid anhydride is tetradecenyl succinic anhydride.

12. Method according to claim 6 wherein the reagent is an oxidizing agent.

13. Method according to claim 12 wherein the oxidizing agent is sodium hypochloride.

14. Method according to claim 6 wherein the reagent is an alkylene oxide.

15. Method according to claim 14 wherein the alkylene oxide is propylene oxide.

16. Method according to claim 1 wherein the at least one active containing material further comprises at least one surfactant.

17. Method according to claim 1, wherein the active is chosen from oils, waxes and petrolatum.

18. Method according to claim 1 wherein the active is a sunscreen, flavor, fragrance or perfume.

19. The dry free flowing powder as produced in claim 1.

20. Personal care composition comprising the dry free flowing powder of claim 19 wherein the personal care composition is a hair or skin composition.

21. Household composition comprising the dry free flowing powder of claim 19 wherein the household composition is chosen from hard surfaces cleansers, room and rug deodorizers and fabric care compositions.
